(19) United States
(12) Patent Application Publication
(10) Pub. No.: US 2022/0272131 A1
(43) Pub. Date: Aug. 25, 2022

(54) METHOD, ELECTRONIC DEVICE AND SYSTEM FOR GENERATING RECORD OF TELEMEDICINE SERVICE

(71) Applicant: PUZZLE AI CO., LTD., Seoul (KR)

(72) Inventors: Ha Rin Jun, Daejeon (KR); Yong-Sik Kim, Seoul (KR); Soon Yong Kwon, Seoul (KR); Gyoungdon Joo, Daegu (KR); Byeongjin Kang, Daejeon (KR); Donghyun Park, Daejeon (KR); Dohyun Kim, Busan (KR)

(21) Appl. No.: 17/254,644

(22) PCT Filed: Sep. 4, 2020
(86) PCT No.: PCT/KR2020/011975

§ 371 Date: Dec. 21, 2020

(51) Int. Cl. H04L 65/1083; G10L 25/78; G10L 17/06; G10L 25/57; G10L 17/02; G06T 1/00; G10L 19/018; G16H 40/67; G16H 80/00

(52) U.S. Cl. CPC H04L 65/1086; G10L 25/78; G10L 17/06; G10L 25/57; G10L 17/02; G06T 1/0021; G10L 19/018; G16H 40/67; G16H 80/00

(57) ABSTRACT

According to an aspect of the present disclosure, a method for generating a record of a telemedicine service in a video call between at least two terminal devices is disclosed. The method includes obtaining authentication information of a user authorized to use the telemedicine service, receiving a sound stream of the video call from a terminal device of the at least two terminal devices, detecting a voice signal from the sound stream, verifying whether the voice signal is indicative of the user based on the authentication information, upon verifying that the voice signal is indicative of the user, continuing the video call to generate the record of the telemedicine service, and upon verifying that the voice signal is not indicative of the user, interrupting the video call.

### TECHNICAL FIELD

The present disclosure relates to a method for generating a record of a telemedicine service in an electronic device. More specifically, the present disclosure relates to a method for generating a record of a telemedicine service of a video call between terminal devices.

### BACKGROUND

In recent years, the use of terminal devices such as smartphones and tablet computers has become widespread. Such terminal devices generally allow voice and video communications over wireless networks. Typically, these devices include additional features or applications, which provide a variety of functions designed to enhance user convenience. For example, a user of a terminal device may perform a video call with another terminal device using a camera, a speaker, and microphone installed in the terminal device.

Recently, the use of a video call between a doctor and a patient has increased. For example, the doctor may consult with the patient via a video call using their terminal devices instead of the patient visiting the doctor's office. However, such a video call may have security issues such as authentication of proper parties allowed to participate in the video call and confidentiality of information exchanged in the video call.

### SUMMARY

The present disclosure relates to verifying whether the voice signal, detected from a sound stream of a video call between at least two terminal devices, is indicative of the user authorized to use the telemedicine service, and determining whether to continue the video call based on the verification result.

According to an aspect of the present disclosure, a method, performed in an electronic device, for generating a record of a telemedicine service in a video call between at least two terminal devices is disclosed. The method includes: obtaining authentication information of a user authorized to use the telemedicine service, receiving a sound stream of the video call from a terminal device of the at least two terminal devices, detecting a voice signal from the sound stream, verifying whether the voice signal is indicative of the user based on the authentication information, upon verifying that the voice signal is indicative of the user, continuing the video call to generate the record of the telemedicine service, and upon verifying that the voice signal is not indicative of the user, interrupting the video call.

According to one embodiment of the present disclosure, in the method for generating the record of the telemedicine service in the video call, the detecting the voice signal from the sound stream includes: sequentially dividing the sound stream into a plurality of frames, selecting a set of a predetermined number of the frames in which a voice is detected among the plurality of frames, and detecting the voice signal from the set of the predetermined number of the frames.

According to one embodiment of the present disclosure, in the method for generating the record of the telemedicine service in the video call, the selecting the set of the predetermined number of the frames includes: detecting next frames in which a voice is detected among the plurality of frames, and updating the set of the predetermined number of the frames by replacing some of the frames in the set of the predetermined number of the frames with the next frames.

According to one embodiment of the present disclosure, in the method for generating the record of the telemedicine service in the video call, the verifying whether the voice signal is indicative of the user includes: obtaining voice features of the voice signal by using a machine-learning based model trained to extract the voice features, and verifying whether the voice signal is indicative of the user based on the voice features.

According to one embodiment of the present disclosure, in the method for generating the record of the telemedicine service in the video call, the authentication information includes voice features of the user, and the verifying whether the voice signal is indicative of the user includes determining a degree of similarity between the obtained voice features and the voice features of the authentication information.

According to one embodiment of the present disclosure, in the method for generating the record of the telemedicine service in the video call, the continuing the video call to generate the record of the telemedicine service comprises includes: generating an image indicative of intensity of the voice signal according to time and frequency, generating a watermark indicative of the voice features, and inserting the watermark into the image.

According to one embodiment of the present disclosure, in the method for generating the record of the telemedicine service in the video call, the continuing the video call to generate the record of the telemedicine service comprises includes: generating voice array data including a plurality of transform values configured to transform the voice signal into a plurality of digital values, generating a watermark indicative of the voice features, and inserting portion of the watermark into the plurality of transform values of the voice array data.

According to one embodiment of the present disclosure, in the method for generating the record of the telemedicine service in the video call, the watermark includes at least one of health information collected from medical devices, a date of medical treatment, a medical treatment number, a patient number, or a doctor number for the authorized user.

According to one embodiment of the present disclosure, in the method for generating the record of the telemedicine service in the video call, the interrupting the video call includes transmitting a command to the terminal device to limit access to the video call.

According to one embodiment of the present disclosure, in the method for generating the record of the telemedicine service in the video call, the interrupting the video call includes transmitting a command to the terminal device to perform authentication of the user.

According to one embodiment of the present disclosure, in the method for generating the record of the telemedicine service in the video call, the method further includes: upon verifying that the voice signal is indicative of the user, generating text corresponding to the voice signal by using speech recognition, and adding at least one portion of the text to the record.

According to another aspect of the present disclosure, an electronic device for generating a record of a telemedicine service in a video call between at least two terminal devices, the electronic device includes a communication circuit configured to communicate with the at least two terminal devices, a memory, and a processor is disclosed. The processor is configured to obtain authentication information of a user authorized to use the telemedicine service, receive a sound stream of the video call from a terminal device of the at least two terminal devices, detect a voice signal from the sound stream, verify whether the voice signal is indicative of the user based on the authentication information, upon verifying that the voice signal is indicative of the user, continue the video call to generate the record of the telemedicine service, and upon verifying that the voice signal is not indicative of the user, interrupt the video call.

According to another aspect of the present disclosure, a system for generating a record of a telemedicine service in a video call is disclosed. The system includes at least two terminal devices configured to perform the video call between the at least two terminal devices, and transmit a sound stream of the video call to an electronic device. The system also includes the electronic device configured to obtain authentication information of a user authorized to use the telemedicine service, receive the sound stream of the video call from a terminal device of the at least two terminal devices, detect a voice signal from the sound stream, verify whether the voice signal is indicative of the user based on the authentication information, upon verifying that the voice signal is indicative of the user, continue the video call to generate the record of the telemedicine service, and upon verifying that the voice signal is not indicative of the user, interrupt the video call.

### BRIEF DESCRIPTION OF DRAWINGS

Embodiments of the inventive aspects of this disclosure will be understood with reference to the following detailed description, when read in conjunction with the accompanying drawings.

FIG. 1A illustrates a system for generating a record of a telemedicine service via a video call according to one embodiment of the present disclosure. FIG. 1B illustrates a system for generating a record of a telemedicine service via a video call according to one embodiment of the present disclosure.

FIG. 2 illustrates a block diagram of an electronic device and a terminal device according to one embodiment of the present disclosure.

FIGS. 3A and 3B illustrate exemplary screenshots of an application for providing the telemedicine service in the terminal devices.

FIG. 4 illustrates a method of verifying whether a voice signal is indicative of a user authorized to use a telemedicine service during a video call according to one embodiment of the present disclosure.

FIGS. 5A and 5B are graphs for illustrating a method of generating an image indicative of intensity of a voice signal according to time and frequency.

FIG. 6 illustrates a voice array data including a plurality of transform values configured to transform the voice signal into a plurality of digital values according to one embodiment of the present disclosure.

FIG. 7 illustrates a flow chart of a method for generating a record of a telemedicine service in a video call between at least two terminal devices in an electronic device according to one embodiment of the present disclosure.

FIG. 8 illustrates a flow chart of a method for generating a record of a telemedicine service in a video call between at least two terminal devices in an electronic device according to another embodiment of the present disclosure.

FIG. 9 illustrates a flow chart of a process of detecting a voice signal from a sound stream according to one embodiment of the present disclosure.

FIG. 10 illustrates a process of selecting a set of a predetermined number of frames from the sound stream according to one embodiment of the present disclosure.

FIG. 11 illustrates a flow chart of a method for generating a record of a telemedicine service in a video call between at least two terminal devices in the electronic device according to still another embodiment of the present disclosure.

FIG. 12 illustrates a flow chart of a process of continuing the video call to generate a record of telemedicine service according to one embodiment of the present disclosure.

FIG. 13 illustrates a flow chart of a process of continuing the video call to generate a record of telemedicine service according to one embodiment of the present disclosure.

### DETAILED DESCRIPTION

Reference will now be made in detail to various embodiments, examples of which are illustrated in the accompanying drawings. In the following detailed description, numerous specific details are set forth in order to provide a thorough understanding of the inventive aspects of this disclosure. However, it will be apparent to one of ordinary skill in the art that the inventive aspects of this disclosure may be practiced without these specific details. In other instances, well-known methods, procedures, systems, and components have not been described in detail so as not to unnecessarily obscure aspects of the various embodiments.

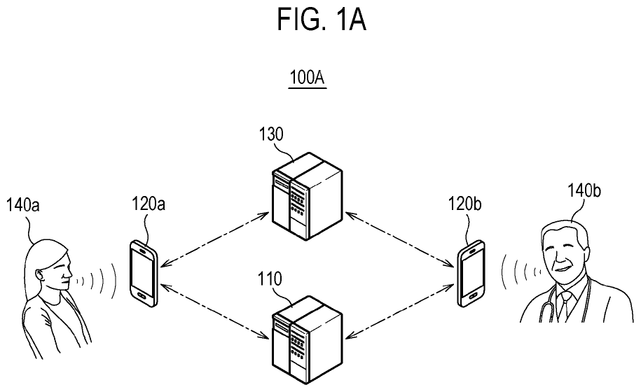

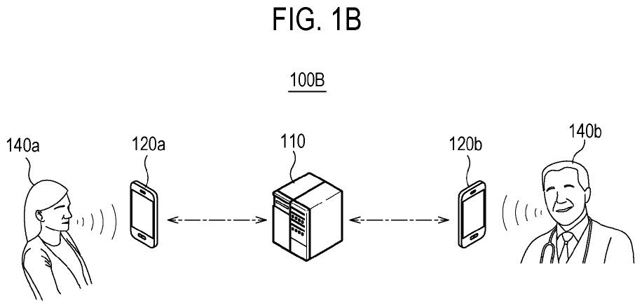

FIG. 1A illustrates a system 100A for generating a record of a telemedicine service via a video call according to one embodiment of the present disclosure. The system 100 includes an electronic device 110, at least two terminal devices 120a and 120b, and a server 130 for generating a record of a telemedicine service. The terminal devices 120a and 120b and the electronic device 110 may communicate with each other through a wireless network and/or a wired network. The terminal devices 120a and 120b and the server 130 may also communicate with each other through a wireless network and/or a wired network. The terminal devices 120a and 120b may be located in different geographic locations.

In the illustrated embodiment, the terminal devices 120a and 120b are presented only by way of example, and thus the number of terminal devices and the location of each of the terminal devices may be changed. The terminal devices 120a and 120b may be any suitable device capable of sound and/or video communication such as a smartphone, cellular phone, laptop computer, tablet computer, or the like.

The terminal devices 120a and 120b may perform a video call with each other through the server 130. The video call between the terminal devices 120a and 120b may be related to a telemedicine service. For example, a user 140a of the terminal device 120a may be a patient and a user 140b of the terminal device 120b may be his or her doctor. The user 140b of the terminal device 120b may provide a telemedicine service to the user 140a of the terminal device 120a through the video call.

During the voice call, the terminal device 120a may capture a sound stream that includes voice uttered by the user 140a via one or more microphones and an image stream that includes images of the user 140a via one or more cameras. The terminal device 120a may transmit the captured sound stream and image stream as a video stream to the terminal device 120b through the server 130, which may be a video call server. Similarly, the terminal device 120b may operate like the terminal device 120a. The terminal device 120b may capture a sound stream that includes voice uttered by the user 140b (e.g., a doctor, a nurse, or the like) via one or more microphones and an image stream, that includes images of the user 140b via one or more cameras. The terminal device 120b may transmit the captured sound stream and image stream as a video stream to the terminal device 120a through the server 130. In such an arrangement, even if the users 140a and 140b are located in different geographic locations, the users 140a and 140b can use the telemedicine service using the video call.

The electronic device 110 may verify whether the users 140a and 140b participating in the video call are authorized to use the telemedicine service. Initially, the electronic device 110 may obtain authentication information of each of the users 140a and 140b from the terminal devices 120a and 120b, respectively, and may store the obtained authentication information. For example, the authentication information of the user 140a may include voice features of the user 140a. The terminal device 120a may display a message on a display screen and prompt the user 140a to read a predetermined phrase so that the voice of the user 140a is processed to generate acoustic features thereof. In one embodiment, the voice features of the user's voice may be generated. The terminal device 120a may transmit to electronic device 110 authentication information of the user 140a authorized to use the telemedicine service. According to another embodiment of the present disclosure, the electronic device 110 may receive a sound stream including the user's voice related to the predetermined phrase from the terminal device 120a, and process the sound stream to generate the authentication information of the user 140a. Similarly, the terminal device 120b may operate like the terminal device 120a.

The electronic device 110 may receive a sound stream of the video call, which is transmitted from the terminal device of the at least one two terminal device 120a and 120b. The electronic device 110 may receive the sound stream of the video call in real time during the video call between the at least two terminal devices 120a and 120b. In one embodiment, the terminal device 120a may extract a sound stream from the video stream of the video call between the at least two terminal devices 120a and 120b. The terminal device 120a may transmit the extracted sound stream to electronic device 110. In this case, the terminal device 120a may transmit the image stream and the sound stream of the video call generated by the terminal device 120a to the server 130, and may transmit only the sound stream of the video call to the electronic device 110. As used herein, the term “sound stream” refers to a sequence of one or more sound signals or sound data, and the term “image stream” refers to a sequence of one or more image data. The electronic device 110 may receive the sound stream from the terminal device 120a.

Similarly, the electronic device 110 may receive the sound stream, which is transmitted from the terminal device 120b. In one embodiment, the terminal device 120b may extract a sound stream from the video stream of the video call between the at least two terminal devices 120a and 120b. The terminal device 120b may transmit the extracted sound stream to electronic device 110. In this case, the terminal device 120b may transmit the image stream and the sound stream of the video call generated by the terminal device 120b to the server 130, and may transmit only the sound stream of the video call to the electronic device 110.

The electronic device 110 may detect a voice signal from the sound stream. Since the sound stream may include a voice signal and noise, the electronic device 110 may detect the voice signal from the sound stream for user authentication. For detecting a voice signal, any suitable voice activity detection (VAD) methods can be used. For examples, the electronic device 110 may extract a plurality of sound features from the sound stream and determine whether the extracted sound features are indicative of a sound of interest such as human voice by using any suitable sound classification method such as a Gaussian mixture model (GMM) based classifier, a neural network, a hidden Markov model (HMM), a graphical model, a Support Vector Machine (SVM), or the like. The electronic device 110 may detect at least one portion where the human voice is detected in the sound stream. A specific method of detecting the voice from the sound stream will be described later.

According to an embodiment, the electronic device 110 may convert the sound stream, which is an analog signal, into a digital signal through a PCM (pulse code modulation) process, and may detect the voice signal from the digital signal. In this case, the electronic device may detect the voice signal from the digital signal according to a specific sampling frequency determined according to a preset frame rate. The PCM process may include a sampling step, a quantizing step, and an encoding step. In addition to the PCM process, various analog-to-digital conversion methods may be used. According to another embodiment, the electronic device 110 may detect the voice signal from the sound stream, which is an analog signal.

The electronic device 110 may verify whether the voice signal is indicative of an actual voice uttered by a person. That is, the electronic device 110 may verify whether the voice signal relates to an actual voice uttered by a person or relates to a recorded voice of a person. The electronic device 110 may distinguish between the voice signal related to the actual voice uttered by a person and the voice signal related to the recorded voice of a person by using a suitable voice spoofing detection method. In one embodiment, the electronic device 110 may perform voice spoofing detection by extracting voice features from the voice signal, and verifying, by using a machine-learning based model, whether the extracted voice features of the voice signal are indicative of an actual voice uttered by a person. For example, the electronic device 110 may extract the voice features by applying a suitable feature extraction algorithm such as a Mel-Spectrogram, Mel-filterbank, MFCC (Mel-frequency cepstral coefficient), or the like. In one embodiment, the electronic device 110 may store a machine-learning based model trained to detect a difference between a recorded voice and an actual voice of a person. For example, the machine-learning based model may include an RNN (recurrent neural network) model, a CNN (convolutional neural network) model, a TDNN (time-delay neural network) model, an LSTM (long short term memory) model, or the like.

If the voice signal is determined not to be indicative of an actual voice uttered by a person, the electronic device 110 may interrupt the video call. On the other hand, if the voice signal is determined to be indicative of an actual voice uttered by a person, the electronic device 110 may verify whether the voice signal included in the sound stream of the video call is indicative of a user (e.g., user 140a or 140b) authorized to use the telemedicine service based on the authentication information. Initially, the electronic device 110 may analyze a voice frequency of the voice signal. Based on the analysis, the electronic device 110 may generate an image (e.g., a spectrogram) indicative of intensity of the voice signal according to time and frequency. A specific method of generating such an image will be described later.

The electronic device 110 may obtain voice features based on the voice signal. For example, the electronic device 110 may store a machine-learning based model trained to extract voice features corresponding to a voice signal. The electronic device 110 may train the machine-learning based model to output voice features from the voice signal input to the machine-learning based model. The machine-learning based model may include an RNN (recurrent neural network) model, a CNN (convolutional neural network) model, a TDNN (time-delay neural network) model, an LSTM (long short term memory) model, or the like. The electronic device 110 may input the voice signal to the machine-learning based model, and may obtain the extracted voice features indicative of the voice signal from the machine-learning based model.

According to another embodiment of the present disclosure, the electronic device 110 may obtain voice features based on the image indicative of intensity of the voice signal according to time and frequency. In this case, the machine-learning based model may be trained to extract voice features corresponding to such an image. The electronic device 110 may train the machine-learning based model to output voice features from an image when the image is input to the machine-learning based model. The electronic device 110 may input the image to the machine-learning based model, and may obtain the extracted voice features indicative of the voice signal from the machine-learning based model.

In one embodiment, the voice features extracted from the machine-learning based model may be feature vectors representing unique voice features of a user. For example, the voice features may be a D-vector extracted from the RNN model. In this case, the electronic device 110 may process the D-vector to generate a matrix or array of hexadecimal alphabet and number combinations. The electronic device 110 may process the D-vector in the form of a UUID (universal unique identifier) used for software construction. The UUID is an identifier standard that does not overlap between identifiers, and may be an identifier optimized for voice identification of users.

According to an embodiment of the present disclosure, the electronic device 110 may generate a private key corresponding to the voice features. The private key may be a key generated by encrypting the voice features, e.g., the D-vector and may represent a key encrypted with the voice of a user (e.g., user 140a or 140b). Further, the private key can be used to generate a watermark indicative of the voice features.

The electronic device 110 may verify whether the voice signal is indicative of a user authorized to use the telemedicine service based on the voice features extracted from the voice signal. The electronic device 110 may determine a degree of similarity between the extracted voice features and the voice features of the authentication information of the user by comparing the extracted voice features of the voice signal and the voice features of the authentication information of the user. For example, the electronic device 110 may determine the degree of similarity by using an edit distance algorithm. The edit distance algorithm, as an algorithm for calculating the degree of similarity of two strings, may be an algorithm that determines the degree of similarity by comparing the number of times insertion, deletion, and change between the two strings. In this case, the electronic device 110 may calculate the degree of similarity between the voice features extracted from the voice signal and the voice features of the authentication information of the user, by applying the voice features extracted from the voice signal and the voice features of the authentication information of the user to the edit distance algorithm. For example, the electronic device 110 may calculate the degree of similarity between a D-vector representing the extracted voice features and a D-vector representing the voice features of the authentication information of the user by using the edit distance algorithm.

With reference to FIG. 1A, the electronic device 110 may determine the degree of similarity between the voice signal detected from the sound stream received from the terminal device 120a, and the voice features of the authentication information of the user 140a. The degree of similarity is then compared to a predetermined threshold value. If the degree of similarity exceeds the predetermined threshold value, the electronic device 110 may determine that the voice signal is indicative of the user 140a. If the degree of similarity does not exceed the predetermined threshold value, the electronic device 110 may determine that the voice signal is not indicative of the user 140a.

Similarly, the electronic device 110 may also determine the degree of similarity between the voice signal detected from the sound stream received from the terminal device 120b, and the voice features of the authentication information of the user 140b. The degree of similarity is then compared to a predetermined threshold value. If the degree of similarity exceeds the predetermined threshold value, the electronic device 110 may determine that the voice signal is indicative of the user 140b. If the degree of similarity does not exceed the predetermined threshold value, the electronic device 110 may determine that the voice signal is not indicative of the user 140b.

The electronic device 110 may determine whether to continue the video call based on the verification result. Upon verifying that the voice signal is indicative of the user, the electronic device 110 may continue the video call to generate the record of the telemedicine service. On the other hand, if the voice signal is determined not to be indicative of the user, the electronic device 110 may interrupt the video call to limit access to the video call by the terminal devices 120a and/or 120b.

In an embodiment, upon verifying that the voice signal is indicative of the user, the electronic device may generate and insert a watermark into the image indicative of intensity of the voice signal according to time and frequency. The electronic device 110 may generate the watermark corresponding to the voice features if the voice signal is verified to be indicative of the user. For example, the electronic device 110 may generate the watermark by encrypting the voice features using a symmetric encryption scheme that performs encryption and decryption based on the same symmetric key. The symmetric encryption scheme may implement an AES (advanced encryption standard) algorithm. The symmetric key may be the private key corresponding to the voice features (e.g., D-vector) of the authentication information of the user 140a or 140b. In addition to the voice features, the watermark include encrypted medical information described below.

After generating the watermark, the electronic device 110 may insert the watermark into the image. The watermark may include medical information related to the video call, the voice features of the user, and the like. In one embodiment, the medical information may include at least one of user's health information collected from medical devices, a date of medical treatment, a medical treatment number, a patient number, or a doctor number. The medical devices may include, for example, a thermometer, a blood pressure monitor, a smartphone, a smart watch, and the like that are capable of detecting one or more physical or medical signals or symptoms and communicating with the terminal device 120a or 120b. In addition, the information included in the watermark may be encrypted using the symmetric encryption scheme.

The electronic device 110 may insert a watermark or a portion thereof into selected pixels among a plurality of pixels included in the image. The electronic device 110 may extract RGB values for each of the plurality of pixels included in the image, and select at least one pixel to insert the watermark based on the RGB values. For example, the electronic device 110 may calculate a difference between the extracted RGB value and the average value of the RGB values for all pixels for each of the plurality of pixels. The electronic device 110 may then select at least one pixel from among the plurality of pixels whose calculated difference is less than a predetermined threshold. In this case, since the electronic device 110 may insert the watermark by selecting the at least one pixel with less color modulation among the plurality of the pixels, it is possible to minimize the modulation of the image. That is, the selected at least one pixel may indicate a pixel of low importance in the method of verifying the user by using the image indicative of the voice signal.

In another embodiment, upon verifying that the voice signal is indicative of the user, the electronic device 110 may insert a watermark into a voice array data. The electronic device 110 may generate voice array data including a plurality of transform values configured to transform the voice signal into a plurality of digital values. The electronic device 110 may insert a portion of the watermark into each of the plurality of transform values of the voice array data. A specific method of inserting the watermark in the voice array data will be described later.

On the other hand, upon verifying that the voice signal is not indicative of the user, the electronic device 110 may interrupt the video call. In this case, the electronic device 110 may transmit a command to at least one of the at least two terminal devices 120a and 120b to limit access to the video call. The command to the terminal device may be a command to perform authentication of the user. In response to the command, the terminal device 120a or 120b may perform authentication of the user 140a or 140b by requiring the user 140a or 140b to input an ID/password, fingerprint, facial image, iris image, or voice.

After completing the video call, the electronic device 110 may convert the image in which the watermark is inserted into a voice file. For example, the electronic device 110 may convert the voice array data in which the watermark is inserted into a voice file. The voice file may be a file having a suitable audio file format such as WAV, MP3, or the like. The electronic device 110 may store the voice file having the audio file format as a record of the telemedicine service.

FIG. 1B illustrates a system 1008 including an electronic device 110 and at least two terminal devices 120a and 120b, and is configured to generate a record of a telemedicine service according to one embodiment of the present disclosure. In this embodiment, the electronic device 110, in addition to performing its functions described with reference to FIG. 1A, may also perform the functions of the server 130 described with reference to FIG. 1A. Thus, the two terminal devices 120a and 120b may perform a video call through the electronic device 110 with the server 130 in FIG. 1A omitted.

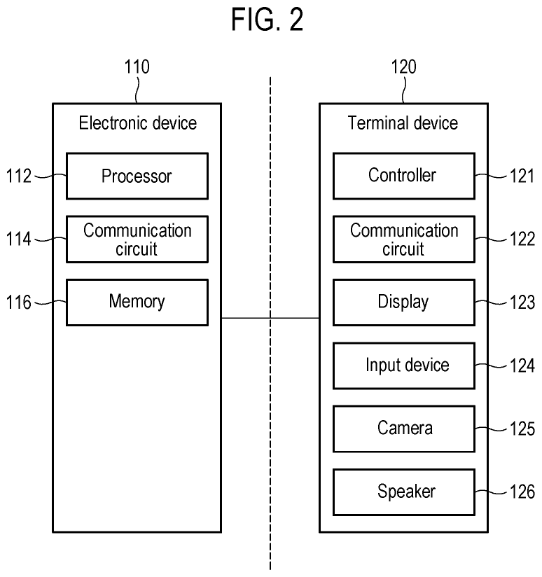

FIG. 2 illustrates a more detailed block diagram of the electronic device 110 and a terminal device 120 (e.g., terminal device 120a and 120b) according to one embodiment of the present disclosure. As shown in FIG. 2, the electronic device 110 includes a processor 112, a communication circuit 114, and a memory 116, and may be any suitable computer system such as a server, web server, or the like.

The processor 112 may execute software to control at least one component of the electronic device 110 coupled with the processor 112, and may perform various data processing or computation. The processor 112 may be a central processing unit (CPU) or an application processor (AP) for managing and operating the electronic device 110.

The communication circuit 114 may establish a direct communication channel or a wireless communication channel between the electronic device 110 and an external electronic device (e.g., the terminal device 120) and perform communication via the established communication channel. For example, the processor 112 may receive authentication information of a user authorized to use the telemedicine service from the terminal device 120 via the communication circuit 114. According to another embodiment of the present disclosure, the processor 112 may receive a sound stream including a user's voice related to a predetermined phrase from the terminal device 120, and process the sound stream to generate the authentication information of the user of the terminal device 120.

Further, the processor 112 may receive a sound stream of a video call from the terminal device 120 via the communication circuit 114. In addition, the communication circuit 114 may transmit various commands from the processor 112 to the terminal device 120.

The memory 116 may store various data used by at least one component (e.g., the processor 112) of the electronic device 110. The memory 116 may include a volatile memory or a non-volatile memory. The memory 116 may store the authentication information of each user. The memory 116 may also store the machine-learning based model trained that can be used to obtain the voice features corresponding to the voice signal. The memory 116 may store the machine-learning based model trained to detect a difference between a recorded voice and an actual voice of a person.

As shown in FIG. 2, the terminal device 120 includes a controller 121, a communication circuit 122, a display 123, an input device 124, a camera 125, and a speaker 126. The configuration and functions of the terminal device 120 disclosed in FIG. 2 may be the same as those of each of the two terminal devices 120a and 120b illustrated in FIGS. 1A and 1B.

The controller 121 may execute software to control at least one component of the terminal device 120 coupled with the controller 121, and may perform various data processing or computation. The controller 121 may be a central processing unit (CPU) or an application processor (AP) for managing and operating the terminal device 120.

The communication circuit 122 may establish a direct communication channel or a wireless communication channel between the terminal device 120 and an external electronic device (e.g., the electronic device 110) and perform communication via the established communication channel. The communication circuit 122 may transmit authentication information of a user authorized to use the telemedicine service from the controller 121 to the electronic device 110. Further, the communication circuit 122 may transmit a sound stream of the video call from the controller 121 to the electronic device 110. In addition, the communication circuit 122 may provide to the controller 121 various commands received from the electronic device 110.

The terminal device 120 may visually output information on the display 123. The display 123 may include touch circuitry adapted to detect a touch, or sensor circuit adapted to detect the intensity of force applied by the touch. The input device 124 may receive a command or data to be used by one or more other components (e.g., the controller 121) of the terminal device 120, from the outside of the terminal device 120. The input device 124 may include, for example, a microphone, touch display, etc.

The camera 125 may capture a still image or moving images. According to an embodiment, the camera 125 may include one or more lenses, image sensors, image signal processors, or flashes. The speaker 126 may output sound signals to the outside of the terminal device 120. The speaker 126 may be used for general purposes, such as playing multimedia or playing record.

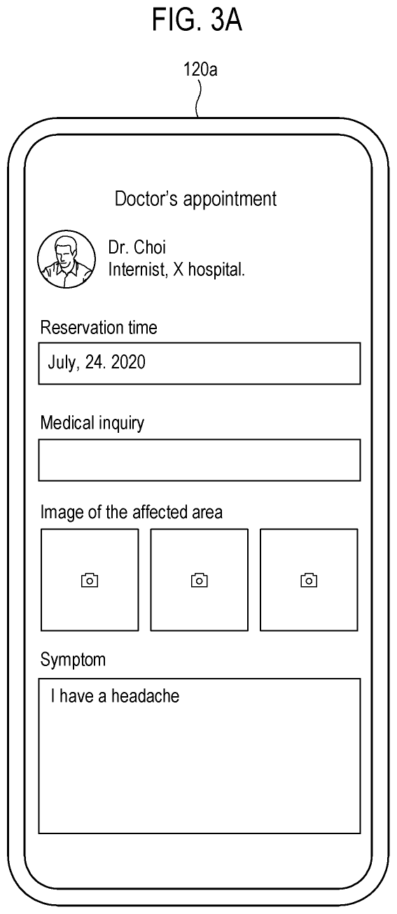

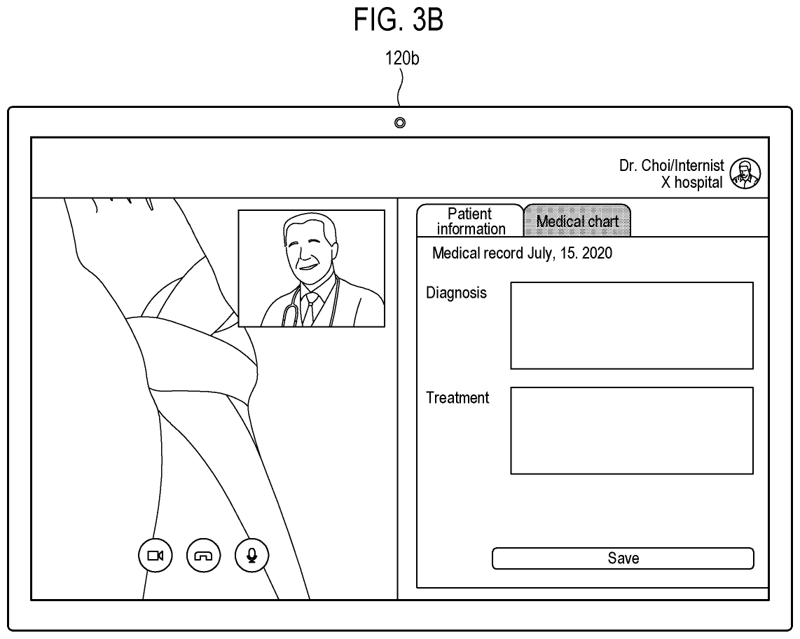

FIGS. 3A and 3B illustrate exemplary screenshots of an application for providing the telemedicine service in the terminal devices 120a and 120b, respectively. In one embodiment, FIG. 3A illustrates a screenshot for making a reservation to use the telemedicine service in the terminal device 120a. The user 140a, for example, a patient, of the terminal device 120a may reserve a video call for telemedicine service with the user 140b, for example, a doctor, of the terminal device 120b. The user 140a of the terminal device 120a may input a reservation time, a medical inquiry, at least one image of the affected area, and a symptom through the application in advance of the video call.

The terminal device 120a may receive a touch input for inputting the symptom of the user 140a through the display 123 or a sound stream including a voice signal uttered by the user 140a through the microphone. When the sound stream including the voice signal uttered by the user 140a is received, the terminal device 120a may transmit the sound stream to the electronic device 110.

The electronic device 110 may verify whether the voice signal is indicative of the user 140a based on the authentication information of the user 140a. If the voice signal is verified to be indicative of the user 140a, the electronic device 110 may generate an image indicative of intensity of the voice signal according to time and frequency, and generate a watermark based on the image. The electronic device 110 may insert the watermark into the image. The electronic device 110 may store the verification result with the voice file obtained by converting the image into which the watermark is inserted. The electronic device 110 may convert the voice array data in which the watermark is inserted into a voice file, and may store the voice file having the audio file format with the verification result.

Upon verifying that the voice signal is indicative of the user 140a, the electronic device 110 may generate text corresponding to the voice signal by using speech recognition. For example, during the voice call, the electronic device 110 may receive the sound stream including the voice signal related to the symptom of the user 140a from the terminal device 120a. In this case, the electronic device 110 may generate text corresponding to the voice signal of the user 140a that relates, for example, to the symptom, by using speech recognition. For generating the text corresponding to the voice signal, any suitable speech recognition methods may be used.

The electronic device 110 may add at least one portion of the text generated from the voice signal to a record of a telemedicine service. For example, the electronic device 110 may transmit the text to the terminal device 120a or 120b. The terminal device 120a or 120b may receive a user input for selecting at least one portion of the text to be added in the record. If the user 140a or 140b selects all portions of the text, the electronic device 110 may add all of the text to the record. If the user 140a or 140b selects one or more specific portions of the text, the electronic device 110 may add the selected specific portions to the record. The electronic device 110 may store the at least one portion of the text corresponding to the voice signal, the voice file obtained by converting the image into which the watermark is inserted, and the verification result as the record. That is, by storing one or more portions of the text related to the voice signal of the user 140a that relates to the symptom by using speech recognition, the record provides facilitates fast and efficient access to and review of relevant information of the telemedicine service.

FIG. 3B illustrates a screenshot for performing the video call for telemedicine service in the terminal device 120b. The users 140a and 140b of terminal devices 120a and 120b, respectively, may perform the video call with each other for the telemedicine service. For example, the user 140a of the terminal device 120a may show his or her affected area (e.g., an image of a foot) to the user 140b of the terminal device 120b, and may explain his or her symptoms to the user 140b during the video call. The user 140b can also show his or her image to the user 140a and explain the diagnosis and treatment contents during the video call.

During the video call, the terminal device 120b may receive a touch input for inputting diagnosis and treatment contents from the user 140b through the touch display or a sound stream including a voice signal uttered by the user 140b through the microphone. When the sound stream including the voice signal uttered by the user 140b is received, the terminal device 120b may transmit the sound stream to the electronic device 110 in real time. The electronic device 110 may verify, in real time, whether the voice signal is indicative of the user 140b based on the authentication information of the user 140b.

If the voice signal is verified to be indicative of the user 140b, the electronic device 110 may generate an image indicative of intensity of the voice signal according to time and frequency, and generate a watermark based on the image. The electronic device 110 may insert the watermark into the image. The electronic device 110 may store the verification result with the voice file obtained by converting the image into which the watermark is inserted. If the voice signal is verified not to be indicative of the user 140b, the electronic device 110 may interrupt the video call. During the video call, the terminal device 120a may also perform operations and functions that are similar to those of the terminal device 120b and communicate with the electronic device 110. Thus, the electronic device 110 may communicate with both terminal devices 120a and 120b simultaneously during the video call.

Upon verifying that the voice signal is indicative of the user 140b, the electronic device 110 may generate text corresponding to the voice signal by using speech recognition. For example, during the voice call, the electronic device 110 may receive the sound stream including the voice signal of the user 140b that relates to diagnosis and treatment of the symptom of the user 140a from the terminal device 120b. In this case, the electronic device 110 may generate text corresponding to the diagnosis and treatment contents using a suitable speech recognition method.

The electronic device 110 may add at least one portion of the text generated from the voice signal to the same record of the telemedicine service with the user 140a or a record, which is separate from that of the user 140a. For example, the electronic device 110 may transmit the text to the terminal device 120b. The terminal device 120b may receive a user input for selecting at least one portion of the text to be added in the record. If the user 140b selects all portions of the text, the electronic device 110 may add all of the text to the record. If the user 140b selects one or more specific portions of the text, the electronic device 110 may add the selected specific portions to the record. The electronic device 110 may store the at least one portion of the text corresponding to the voice signal, the voice file obtained by converting the image into which the watermark is inserted, and the verification result as the record. That is, by storing the text related to the diagnosis and treatment contents using speech recognition, the record provides facilitates fast and efficient access to and review of relevant information of the telemedicine service.

In one embodiment of present disclosure, in the case of a sound stream related to some diagnostic contents (e.g., patient information that should not be disclosed), the terminal device 120b may transmit the sound stream only to the electronic device 110, and may not transmit the sound stream to the terminal device 120a. For example, when the user 140b mutes the sound stream delivered to the user 140a and inputs a voice signal related to confidential or sensitive diagnostic information to the terminal device 120b, the terminal device 120b may transmit the sound stream related to such diagnostic contents only to the electronic device 110.

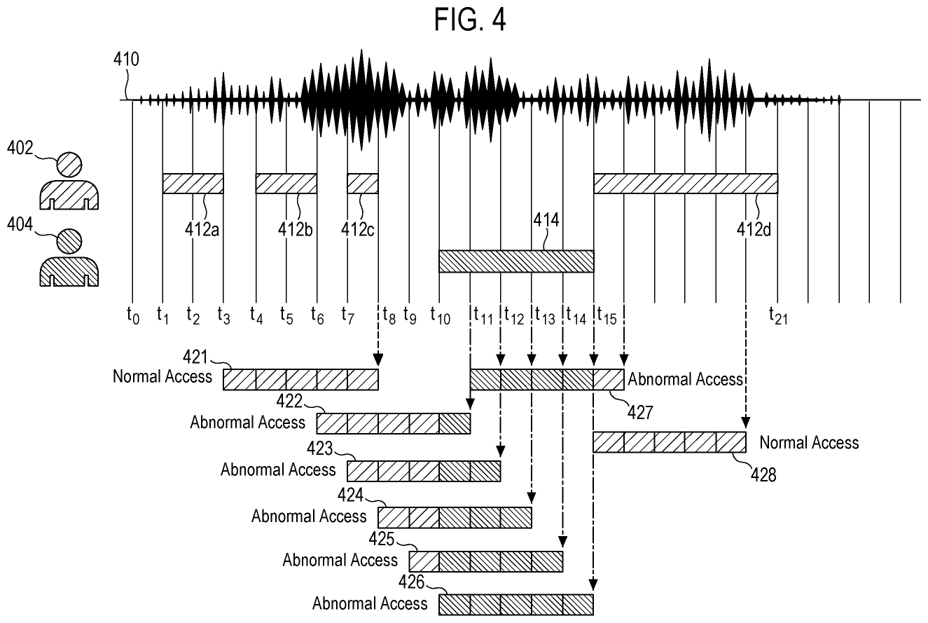

FIG. 4 illustrates a method of verifying whether a voice signal is indicative of a user authorized to use a telemedicine service during a video call according to one embodiment of the present disclosure. In one embodiment, the electronic device 110 may receive a sound stream 410 from a terminal device 120a or 120b. The sound stream 410 may contain the voices of two users 402 and 404 from one of the terminal devices 120a or 120b. In this case, the user 402 is a user authorized to use the telemedicine service, and the user 404 is not a user authorized to use the telemedicine service. When a voice of the user 402 is detected in the sound stream 410, the electronic device 110 may verify that the voice of the user 402 is indicative of the authorized user and thus determine that the access is normal access to the telemedicine service. On the other hand, when a voice of the user 404 is detected in the sound stream 410, the electronic device 110 may verify that the voice of the user 404 is not indicative of the authorized user and thus determine that the access is an abnormal access to the telemedicine service.

For verifying whether a voice signal is indicative of an authorized user, a voice signal of a predetermined period of time (e.g., 5 sec) may be sequentially captured and processed. For example, the electronic device 110 may select portions of the sound stream for the predetermined period of time where the voice signal is detected, and may verify whether the user is authorized to use the telemedicine service based on the selected portions. In FIG. 4, a voice signal for 5seconds is used for the predetermined period of time. However, the predetermined period of time may be any period of time between 3 to 10 seconds, but is not limited thereto.

The electronic device 110 may sequentially divide the sound stream 410 into a plurality of frames. If the sound stream 410 is converted from its analog signal to a digital signal according to specific sampling frequency determined according to a preset frame rate, the number of frames included in the unit time (e.g., 1 sec) is determined according to the sampling rate. For example, when the sampling rate is 16,000 Hz, 16,000 frames are included in the unit time. That is, for authenticating the voice of a user, 80,000 frames are required.

The electronic device 110 may select a set of a predetermined number of the frames in which a voice is detected among the plurality of frames. The electronic device 110 may select frames in which the human voice is detected at unit time intervals. For example, if the voice is not detected from t₀ to t₁, the electronic device 110 may not select frames included between t₀ to t₁. When the voice is detected from t1 to t3, the electronic device 110 may select frames 412a included between t1 to t3. In this manner, the electronic device 110 may select frames 412a, 412b, and 412c included in time intervals from t₁ to t₃, from t₄ to t₆, and from t₇ to t₈, respectively. In this case, by selecting a set of frames of the predetermined number (e.g., 80,000), a voice signal for the predetermined period of time is obtained.

The electronic device 110 may detect the voice signal 421 from the set of the predetermined number of frames. The electronic device 110 may verify whether the voice signal 421 is indicative of the user 402 based on the authentication information. The electronic device 110 may extract voice features from the voice signal 421, and may determine a degree of similarity between the extracted voice features of the voice signal 421 and the voice features of the authentication information of the user 402. The degree of similarity is compared to a predetermined threshold value. If the degree of similarity exceeds the predetermined threshold value, the electronic device 110 may determine that the voice signal 421 is indicative of the user 402. Since the user 402 is a user who is authorized to use the telemedicine service, the degree of similarity will exceed the predetermined threshold value. Upon the verifying that the voice signal 421 is indicative of the user 402, the electronic device 110 may continue the video call between the terminal devices 120a and 120b.

The set of a predetermined number of the frames may be in the form of a queue. For example, in the set, the frames included in the unit time interval may be input and output in a FIFO (first-in first-out) manner. For example, frames included in the unit time interval may be grouped, and the frames may be input or output to the set.

The electronic device 110 may detect next frames in which voice is detected among the plurality of frames, and may update the set of the predetermined number of frames by replacing some of the frames in the set of the predetermined number of the frames with the next frames. For example, the electronic device 110 may detect a voice in frames included in a time interval from t₁₀ to t₁₁. In this case, the electronic device 110 may replace frames included in the time interval from t₁ to t₂, which are the oldest frames among the set of the predetermined number of the frames, with frames in the newly detected interval from t₁₀ to t₁₁.

The electronic device 110 may detect a voice signal 422 from the updated set of the predetermined number of frames. The electronic device 110 may verify whether the voice signal 422 is indicative of the user 402 based on the authentication information. The electronic device 110 may extract voice features from the voice signal 422, and may determine a degree of similarity between the extracted voice features of the voice signal 422 and the voice features of the authentication information of the user 402. The degree of similarity is compared to a predetermined threshold value. Since the user 404 is not a user who is authorized to use the telemedicine service and the voice signal 422 includes the voice signal of the user 404, the degree of similarity will not exceed the predetermined threshold value. Upon the verifying that the voice signal 422 is not indicative of the user 402, the electronic device 110 may interrupt the video call.

In a similar manner, the electronic device 110 may determine that the voice signals 423, 424, 425, 426, and 427 detected from the updated set of the predetermined number of frames are not indicative of the user 402. In such cases, the electronic device 110 may interrupt the video call.

The electronic device 110 may detect a voice in frames 412d included in a time interval from t₁₅ to t₂₁. In this case, the set may include frames included in time intervals from t₁₅ to t₂₁. The electronic device 110 may detect the voice signal 428 from the set of the predetermined number of frames. The electronic device 110 may verify whether the voice signal 428 is indicative of the user 402 based on the authentication information. Since the user 402 is a user who is authorized to use the telemedicine service, the degree of similarity will exceed the predetermined threshold value. Upon the verifying that the voice signal 428 is indicative of the user 402, the electronic device 110 may continue the video call.

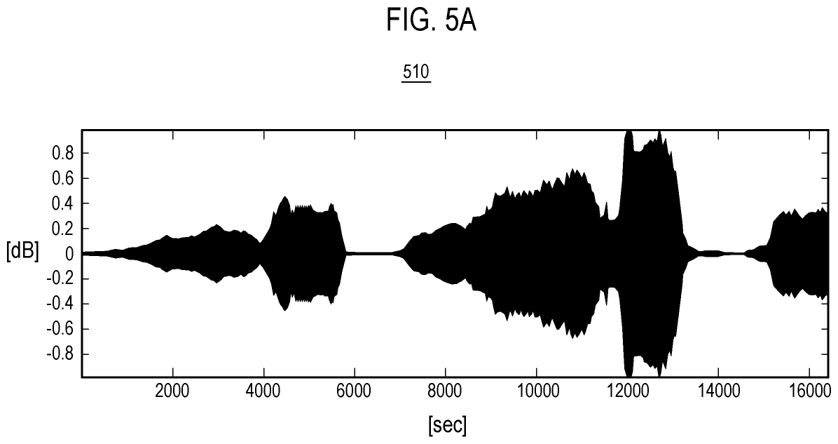

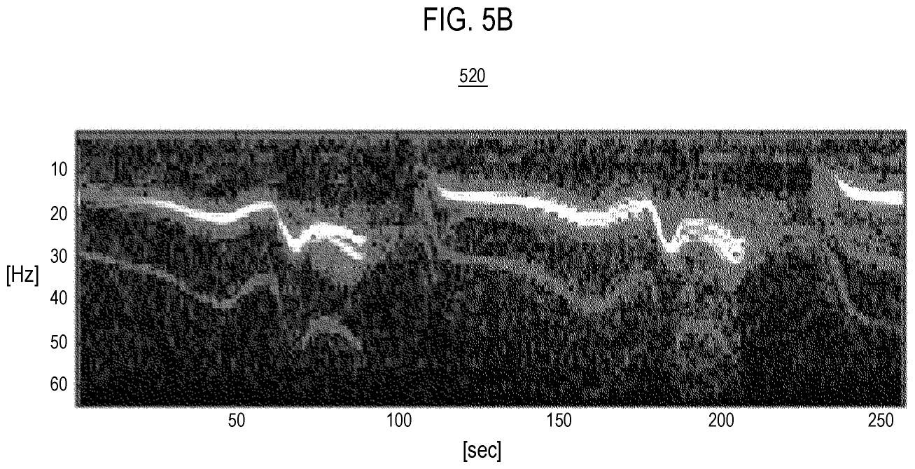

FIGS. 5A and 5B are graphs for illustrating a method of generating an image indicative of intensity of a voice signal according to time and frequency. FIG. 5A illustrates a graph 510 of the voice signal representing amplitude over time, and FIG. 5B is an image 520 indicative of intensity of the voice signal according to time and frequency according to one embodiment of the present disclosure.

The graph 510 represents the voice signal detected from the sound stream. The x-axis of the graph 510 represents time, and the y-axis of the graph 510 represents an intensity of the voice signal. The electronic device 110 may generate an image based on the voice signal.

The electronic device 110 may generate an image 520 including a plurality of pixels indicative of intensity of the voice signal according to time and frequency shown in FIG. 5B by applying the voice signal to an STFT (short-time Fourier transform) algorithm. The electronic device 110 may generate the image 520 by applying a suitable feature extraction algorithm such as a Mel-Spectrogram, Mel-filterbank, MFCC (Mel-frequency cepstral coefficient), or the like. The image 520 may be a spectrogram. The x-axis of the image 520 represents time, the y-axis represents frequency, and each pixel represents the intensity of the voice signal.

The electronic device 110 may insert a watermark or a portion thereof into selected pixels among the plurality of pixels included in the image 520. In one embodiment, the electronic device 110 may extract RGB values for each of the plurality of pixels included in the image 520, and select at least one pixel to insert the watermark or a portion of thereof based on the RGB values. For example, the electronic device 110 may calculate a difference between the extracted RGB value and the average value of the RGB values for all pixels for each of the plurality of pixels in the image. The electronic device 110 may then select at least one pixel from among the plurality of pixels whose calculated difference is less than a predetermined threshold. In this case, since the electronic device 110 may insert the watermark by selecting the at least one pixel with less color modulation among the plurality of the pixels, it is possible to minimize the modulation of the image 520. That is, the selected at least one pixel may indicate a pixel of low importance in the method of verifying the user by using the image 520 indicative of the voice signal.

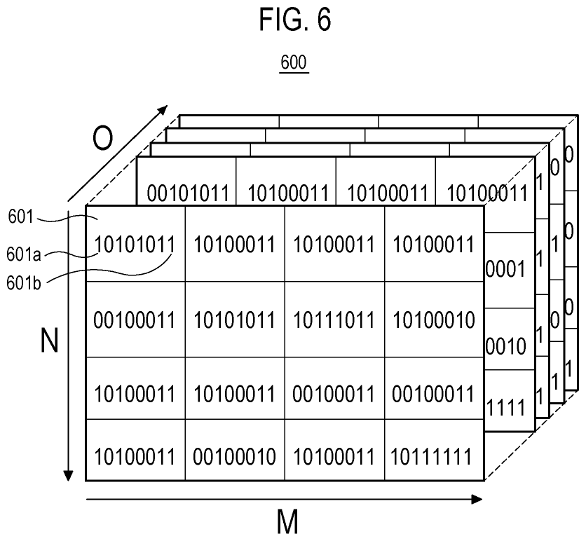

FIG. 6 illustrates a voice array data 600 including a plurality of transform values configured to transform the voice signal into a plurality of digital values according to one embodiment of the present disclosure. The electronic device 110 may generate a plurality of transform values representing the voice signal by converting the voice signal into a digital signal. For example, the electronic device 110 may generate voice array data 600 including the plurality of transform values. The voice array data 600 may have a multidimensional arrangement structure. Referring to FIG. 6, for example, the voice array data 600 may be data in a form in which M×N×O transform values are arranged in a 3-dimensional structure.

The electronic device 110 may insert a portion of a watermark into the plurality of transform values of the voice array data 600. In this case, the watermark may be expressed as a set of digital values of a specific bit included in a matrix of a specific size. For example, the watermark may be a set of 8-bit digital values included in a 16x16 matrix. The electronic device 110 may insert all of the bits included in the watermark into some of the plurality of transform values. The electronic device 110 may insert a portion of the watermark at an LSB (least significant bit) position or an MSB (most significant bit) position of the plurality of transform values. For example, if the all of the bits included in the watermark is 8×16×16, the electronic device 110 may select 8x16x16 transform values among the plurality of transform values, and may insert one bit included in the watermark into the MSB of each of the selected transform values. For example, if a transform value 601 is selected, a portion of the watermark may be inserted in an MSB 601a or LSB 601b of the transform value 601.

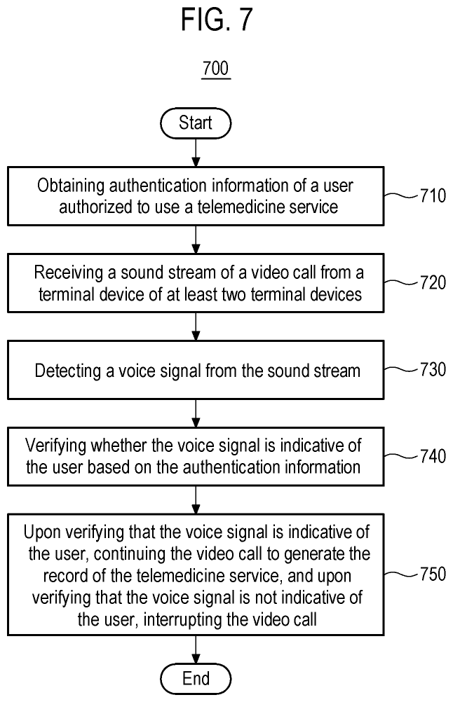

FIG. 7 illustrates a flow chart 700 of a method for generating a record of a telemedicine service in a video call between at least two terminal devices 120a and 120b in an electronic device 110 according to one embodiment of the present disclosure. At 710, the processor 112 of the electronic device 110 may obtain authentication information of the user 140a or 140b authorized to use a telemedicine service. The processor 112 may receive authentication information of the user 140a or 140b from the terminal device 120a or 120b through a communication circuit 114. The processor 112 may store the received authentication information of the user 140a or 140b in the memory 116. When authentication of the user is required, the processor 112 may obtain authentication information of the user 140a or 140b authorized to use the telemedicine service from the memory 116. The authentication information includes voice features (e.g., D-vector) of the user 140a or 140b.

At 720, the processor 112 may receive a sound stream of the video call from a terminal device of the at least two terminal device 120a and 120b. The processor 112 may receive the sound stream of the video call in real-time during the video call between the terminal devices 120a and 120b.

At 730, the processor 112 may detect a voice signal from the sound stream. The processor 112 may detect at least one portion where a human voice is detected in the sound stream by using any suitable voice activity detection (VAD) methods.

At 740, the processor 112 may verify whether the voice signal is indicative of the user 140a or 140b based on the authentication information. In this process, the processor 112 may extract voice features from the voice signal. The processor 112 may determine a degree of similarity between the extracted voice features of the voice signal and the voice features of the authentication information of the user 140a or 140b. The degree of similarity is compared to a predetermined threshold value. If the degree of similarity exceeds the predetermined threshold value, the processor 112 may determine that the voice signal is indicative of the user 140a or 140b. Otherwise, the processor 112 may determine that the voice signal is not indicative of the user 140a or 140b.

At 750, upon verifying that the voice signal is indicative of the user, the processor 112 may continue the video call to generate a record of the telemedicine service, for example, after the completion of the video call or, if the video call is subsequently interrupted for verification failure, up to the time when the voice signal was last verified to be the voice of an authorized user. Upon verifying that the voice signal is not indicative of the user, the processor 112 may interrupt the video call.

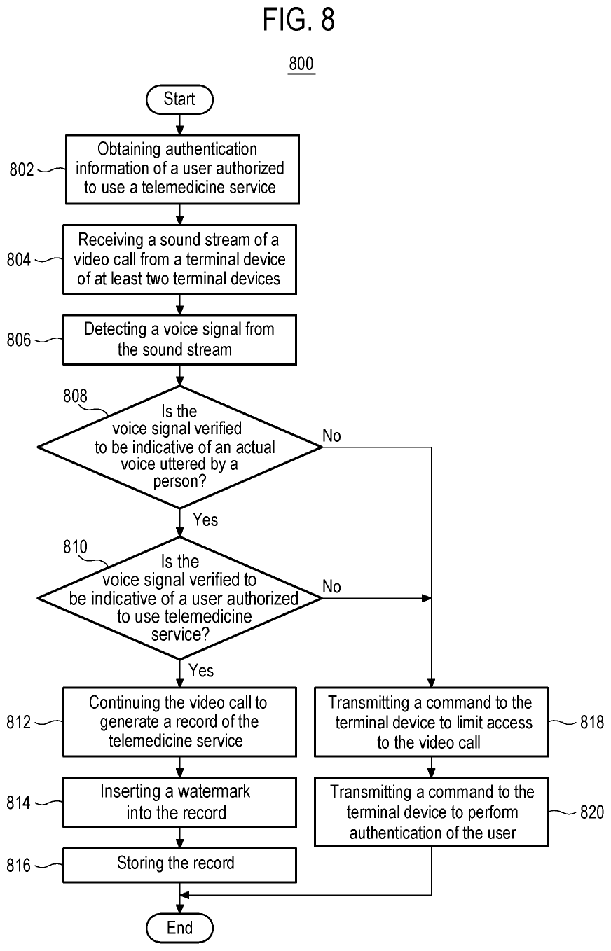

FIG. 8 illustrates a flow chart 800 of a method for generating a record of a telemedicine service in a video call between at least two terminal devices 120a and 120b in an electronic device 110 according to another embodiment of the present disclosure. Descriptions that overlap with those already described in FIG. 7 will be omitted.

At 802, the processor 112 of the electronic device 110 may obtain authentication information of the user 140a or 140b authorized to use the telemedicine service. At 804, the processor 112 may receive a sound stream of a video call from a terminal device of the at least two terminal devices 120a and 120b. At 806, the processor 112 may detect a voice signal from each sound stream.

At 808, the processor 112 may verify whether the voice signal is indicative of an actual voice uttered by a person. The processor 112 may verify whether the voice signal relates to an actual voice uttered by a person or relates to a recorded voice of a person by using a suitable voice spoofing detection method. If the voice signal is verified to be indicative of an actual voice uttered by a person, the method proceeds to 810 where the processor 112 may verify whether the voice signal in each sound stream is indicative of a user authorized to use the telemedicine service. If the voice signal is not verified to be indicative of an actual voice uttered by a person, the method proceeds to 818 where the processor 112 may transmit a command to the terminal device 120a or 120b to limit access to the video call.

At 810, if the voice signal is verified to be indicative of the user 140a or 140b, the method proceeds to 812 where the processor 112 may continue the video call to generate a record of the telemedicine service. At 814, the processor 112 may insert a watermark into the record. At 816, the processor 112 may store the record.

On the other hand, if the voice signal is not verified to be indicative of the user 140a or 140b, the method proceeds to 818 where the processor 112 may transmit a command to the terminal device 120a or 120b to limit access to the video call. At 820, the processor 112 may transmit a command to the terminal device 120a or 120b, from which the voice signal was not verified to be an authorized user was received, to perform authentication of the user. In this case, for resuming the video call, the terminal device 120a or 120b may output an indication on the display or via the speaker for the user to perform authentication. The terminal device 120a or 120b may perform authentication of the user by requiring the user to input an ID/password, fingerprint, facial image, iris image, voice, or the like.

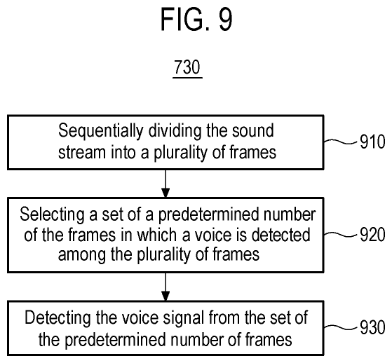

FIG. 9 illustrates a flow chart of the process 730 of detecting a voice signal from the sound stream according to one embodiment of the present disclosure.

At 910, the processor 112 of the electronic device 110 may sequentially divide the sound stream into a plurality of frames. If the sound stream is converted from an analog signal to a digital signal according to specific sampling frequency determined based on a preset frame rate, the number of frames included in the unit time (e.g., 1 sec) is determined according to the sampling rate.

At 920, the processor 112 may select a set of a predetermined number of the frames in which voice is detected among the plurality of frames. In this process, the electronic device 110 may select frames in which human voice is detected at unit time intervals. At 930, the processor 112 may detect the voice signal form the set of the predetermined number of frames.

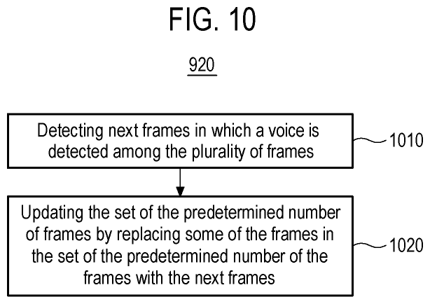

FIG. 10 illustrates the process 920 of selecting a set of a predetermined number of the frames according to one embodiment of the present disclosure.

At 1010, the processor 112 of the electronic device 110 may detect next frames in which a voice is detected among the plurality of frames. The next frames may be frames included in a specific unit time interval in which the voice is detected.

At 1020, the processor 112 may update the set of the predetermined number of frames by replacing some of the frames in the set of the predetermined number of the frames with the next frames.

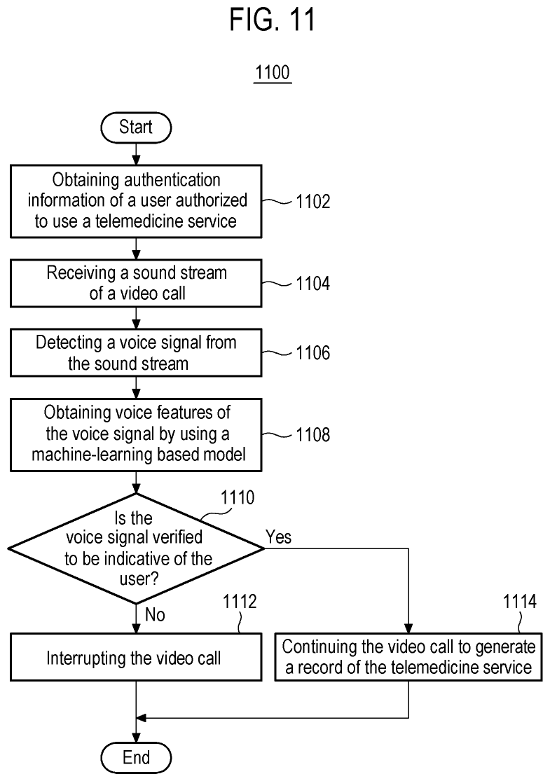

FIG. 11 illustrates a flow chart 1100 of a method for generating a record of a telemedicine service in a video call between at least two terminal devices 120a and 120b in the electronic device 110 according to one embodiment of the present disclosure. Descriptions that overlap with those already described in FIGS. 7 and 8 will be omitted.

At 1102, a processor 112 of the electronic device 110 may obtain authentication information of a user 140a or 140b authorized to use the telemedicine service. At 1104, the processor 112 may receive a sound stream of a video call from a terminal device of the at least two terminal devices 120a and 120b. At 1106, the processor 112 may detect a voice signal from the sound stream.

At 1108, the processor 112 may obtain voice features of the voice signal by using a machine-learning based model. The memory 116 of the electronic device 110 may store a machine-learning based model trained to extract voice features corresponding to a voice signal. The electronic device 110 may train the machine-learning based model to output voice features from a voice signal input to the machine-learning based model. The machine-learning based model may include an RNN (recurrent neural network) model, a CNN (convolutional neural network) model, a TDNN (time-delay neural network) model, an LSTM (long short term memory) model, or the like. The electronic device 110 may input the voice signal detected in the sound stream to the machine-learning based model, and may obtain extracted voice features indicative of the voice signal from the machine-learning based model.

At 1110, the processor 112 may verify whether the voice signal is indicative of the user based on the voice features. If the voice signal is not verified to be indicative of the user, the method proceeds to 1112 where the processor 112 may interrupt the video call. On the other hand, if the voice signal is verified to be indicative of the user, the method proceeds to 1114 where the processor 112 may continue the video call to generate a record of telemedicine service.

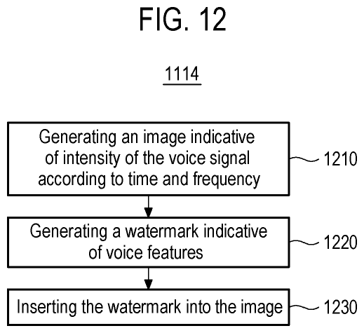

FIG. 12 illustrates the process 1114 of continuing the video call to generate a record of telemedicine service according to one embodiment of the present disclosure. At 1210, the processor 112 may generate an image indicative of intensity of the voice signal according to time and frequency. For example, the electronic device 110 may generate the image by applying the voice signal to an STFT (short-time Fourier transform) algorithm. The electronic device 110 may also generate the image by applying a suitable feature extraction algorithm such as a Mel-Spectrogram, Mel-filterbank, MFCC (Mel-frequency cepstral coefficient), or the like. The image may be a spectrogram.

At 1220, the processor 112 may generate a watermark indicative of the voice features. The processor may then insert the watermark into the image at 1230.

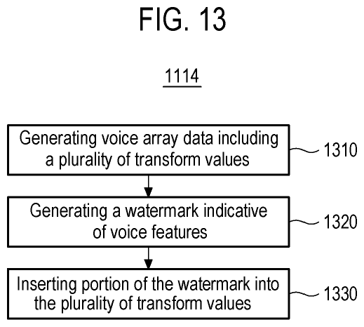

FIG. 13 illustrates the process 1114 of continuing the video call to generate a record of telemedicine service according to one embodiment of the present disclosure. At 1310, the processor 112 may generate voice array data including a plurality of transform values configured to transform the voice signal into a plurality of digital values. The processor 112 may generate the plurality of transform values representing the voice signal by converting the voice signal into a digital signal. The voice array data may have a multidimensional arrangement structure.

At 1320, the processor 112 may generate a watermark indicative of voice features. In this case, the watermark may be expressed as a set of digital values of a specific bit included in a matrix of a specific size. At 1330, the processor 112 may insert one or more portions of the watermark into the plurality of transform values. For example, the processor 112 may insert all of the bits included in the watermark into some of the plurality of transform values. Further, the processor 112 may insert a portion of the watermark at an LSB (least significant bit) position or an MSB (most significant bit) position of the plurality of transform values.

According to an aspect of the present disclosure, an electronic device may verify in real time whether a user who participates in a video call for a telemedicine service is a user authorized to use the telemedicine service. The electronic device may determine whether to continue or interrupt the video call based on the verification result.

According to another aspect of the present disclosure, the electronic device may prevent forgery of medical treatment contents related to the telemedicine service by inserting a watermark into an image related to the voice signal detected from the sound stream of the video call.

In general, the terminal devices described herein may represent various types of devices, such as a smartphone, a wireless phone, a cellular phone, a laptop computer, a wireless multimedia device, a wireless communication personal computer (PC) card, a PDA, or any device capable of video communication through a wireless channel or network. A device may have various names, such as access terminal (AT), access unit, subscriber unit, mobile station, mobile device, mobile unit, mobile phone, mobile, remote station, remote terminal, remote unit, user device, user equipment, handheld device, etc. The devices described herein may have a memory for storing instructions and data, as well as hardware, software, firmware, or combinations thereof.

The techniques described herein may be implemented by various means. For example, these techniques may be implemented in hardware, firmware, software, or a combination thereof. Those of ordinary skill in the art would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, the various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

For a hardware implementation, the processing units used to perform the techniques may be implemented within one or more ASICs, DSPs, digital signal processing devices (DSPDs), programmable logic devices (PLDs), field programmable gate arrays (FPGAs), processors, controllers, micro-controllers, microprocessors, electronic devices, other electronic units designed to perform the functions described herein, a computer, or a combination thereof.

Thus, the various illustrative logical blocks, modules, and circuits described in connection with the disclosure herein are implemented or performed with a general-purpose processor, a DSP, an ASIC, a FPGA or other programmable logic device, discrete gate or transistor logic, discrete hardware components, or any combination thereof designed to perform the functions described herein. A general-purpose processor may be a microprocessor, but in the alternate, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

If implemented in software, the functions may be stored on or transmitted over as one or more instructions or code on a computer-readable medium. Computer-readable media include both computer storage media and communication media including any medium that facilitates the transfer of a computer program from one place to another. A storage media may be any available media that can be accessed by a computer. By way of example, and not limited thereto, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Further, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc, where disks usually reproduce data magnetically, while discs reproduce data optically with lasers. Combinations of the above should also be included within the scope of computer-readable media.

The previous description of the disclosure is provided to enable any person skilled in the art to make or use the disclosure. Various modifications to the disclosure will be readily apparent to those skilled in the art, and the generic principles defined herein are applied to other variations without departing from the spirit or scope of the disclosure. Thus, the disclosure is not intended to be limited to the examples described herein but is to be accorded the widest scope consistent with the principles and novel features disclosed herein.

Although exemplary implementations are referred to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices may include PCs, network servers, and handheld devices.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method, performed in an electronic device, for generating a record of a telemedicine service in a video call between at least two terminal devices, the method comprising:
obtaining authentication information of a user authorized to use the telemedicine service;
receiving a sound stream of the video call from a terminal device of the at least two terminal devices;
detecting a voice signal from the sound stream;
verifying whether the voice signal is indicative of the user based on the authentication information;
upon verifying that the voice signal is indicative of the user, continuing the video call to generate the record of the telemedicine service; and
upon verifying that the voice signal is not indicative of the user, interrupting the video call.

2. The method of claim 1, wherein detecting the voice signal from the sound stream comprises:
sequentially dividing the sound stream into a plurality of frames;
selecting a set of a predetermined number of the frames in which a voice is detected among the plurality of frames; and
detecting the voice signal from the set of the predetermined number of the frames.

3. The method of claim 2, wherein selecting the set of the predetermined number of the frames comprises:
detecting next frames in which a voice is detected among the plurality of frames; and
updating the set of the predetermined number of the frames by replacing some of the frames in the set of the predetermined number of the frames with the next frames.

4. The method of claim 1, wherein verifying whether the voice signal is indicative of the user comprises:
obtaining voice features of the voice signal by using a machine-learning based model trained to extract the voice features; and
verifying whether the voice signal is indicative of the user based on the voice features.

5. The method of claim 4, wherein the authentication information includes voice features of the user, and
wherein verifying whether the voice signal is indicative of the user comprises determining a degree of similarity between the obtained voice features and the voice features of the authentication information.

6. The method of claim 4, wherein continuing the video call to generate the record of the telemedicine service comprises:
generating an image indicative of intensity of the voice signal according to time and frequency;
generating a watermark indicative of the voice features; and
inserting the watermark into the image.

7. The method of claim 4, wherein continuing the video call to generate the record of the telemedicine service comprises:
generating voice array data including a plurality of transform values configured to transform the voice signal into a plurality of digital values;
generating a watermark indicative of the voice features; and
inserting portion of the watermark into the plurality of transform values of the voice array data.

8. The method of claim 6, wherein the watermark comprises at least one of health information collected from medical devices, a date of medical treatment, a medical treatment number, a patient number, or a doctor number for the authorized user.

9. The method of claim 1, wherein interrupting the video call comprises:
transmitting a command to the terminal device to limit access to the video call; and
transmitting a command to the terminal device to perform authentication of the user.

10. The method of claim 1, further comprising:
generating, upon verifying that the voice signal is indicative of the user, text corresponding to the voice signal by using speech recognition; and
adding at least one portion of the text to the record.

11. An electronic device for generating a record of a telemedicine service in a video call between at least two terminal devices, the electronic device comprising:
a communication circuit configured to communicate with the at least two terminal devices;
a memory; and
a processor configured to:
obtain authentication information of a user authorized to use the telemedicine service,
receive a sound stream of the video call from a terminal device of the at least two terminal devices,
detect a voice signal from the sound stream,
verify whether the voice signal is indicative of the user based on the authentication information,
upon verifying that the voice signal is indicative of the user, continue the video call to generate the record of the telemedicine service, and
upon verifying that the voice signal is not indicative of the user, interrupt the video call.

12. The electronic device of claim 11, wherein the processor further configured to:
sequentially divide the sound stream into a plurality of frames,
select a set of a predetermined number of the frames in which a voice is detected among the plurality of frames, and
detect the voice signal from the set of the predetermined number of the frames.

13. The electronic device of claim 12, wherein the processor further configured to:
detect next frames in which a voice is detected among the plurality of frames, and
update the set of the predetermined number of the frames by replacing some of the frames in the set of the predetermined number of the frames with the next frames.

14. The electronic device of claim 11, wherein the processor further configured to:
obtain voice features of the voice signal by using a machine-learning based model trained to extract the voice features, and
verify whether the voice signal is indicative of the user based on the voice features.

15. The electronic device of claim 14, wherein the authentication information includes voice features of the user, and
wherein the processor further configured to determine a degree of similarity between the obtained voice features and the voice features of the authentication information.

16. The electronic device of claim 14, wherein the processor further configured to:
upon verifying that the voice signal is indicative of the user, generate an image indicative of intensity of the voice signal according to time and frequency,
generate a watermark indicative of the voice features, and
insert the watermark into the image.

17. The electronic device of claim 14, wherein the processor further configured to:
upon verifying that the voice signal is indicative of the user, generate voice array data including a plurality of transform values configured to transform the voice signal into a plurality of digital values,
generate a watermark indicative of the voice features, and
insert portion of the watermark into the plurality of transform values of the voice array data.

18. The electronic device of claim 16, wherein the watermark comprises at least one of health information collected from medical devices, a date of medical treatment, a medical treatment number, a patient number, or a doctor number for the authorized user.

19. The electronic device of claim 11, wherein the processor further configured to:
transmit, upon verifying that the voice signal is not indicative of the user, a command to the terminal device to limit access to the video call, and
transmit a command to the terminal device to perform authentication of the user.

20. The electronic device of claim 11, wherein the processor further configured to:
generate, upon verifying that the voice signal is indicative of the user, text corresponding to the voice signal by using speech recognition, and
add at least one portion of the text to the record.

21-30. (canceled)
